(19) United States
(12) Patent Application Publication
(10) Pub. No.: US 2008/0262597 A1
(43) Pub. Date: Oct. 23, 2008

(54) Prosthesis Fixation Apparatus and Methods

(75) Inventors: Jia Hua Xiao, Santa Rosa, CA (US); Brennan Marilla, Santa Rosa, CA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 11/736,453

(22) Filed: Apr. 17, 2007

(51) Int. Cl. A61F 2/06

(52) U.S. Cl. 623/1.15; 623/1.13; 623/1.36

(57) ABSTRACT

Apparatus and methods for endoluminally advancing a tubular prosthesis and a plurality of fasteners to a site in a lumen in a human body and passing the fasteners from an inner surface of the prosthesis through the prosthesis and a wall of the lumen to secure the prosthesis to the wall. Embodiments include simultaneous deployment of fasteners using a graft alone or in conjunction with a stent graft. Another arrangement includes guide lines to guide the end of the fixation deployment device to a specific location of the wall of the prosthesis.

### FIELD OF THE INVENTION

The invention relates to prosthesis fixation in a passageway in a human body such as an artery.

### BACKGROUND OF THE INVENTION

Tubular prostheses such as stents, grafts, and stent-grafts (e.g., stents having an inner and/or outer covering comprising graft material and which may be referred to as covered stents) have been used to treat abnormalities in passageways in the human body. In vascular applications, these devices often are used to replace or bypass occluded, diseased or damaged blood vessels such as stenotic or aneurysmal vessels. For example, it is well known to use stent-grafts, which comprise biocompatible graft material (e.g., Dacron® or expanded polytetrafluoroethylene (ePTFE)) supported by a framework (e.g., one or more stent or stent-like structures), to treat or isolate aneurysms. The framework provides mechanical support and the graft material or liner provides a blood barrier.

Aneurysms generally involve abnormal widening of a duct or canal such as a blood vessel and generally appear in the form of a sac formed by the abnormal dilation of the duct or vessel wall. The abnormally dilated wall typically is weakened and susceptible to rupture. Aneurysms can occur in blood vessels such as in the abdominal aorta where the aneurysm generally extends below the renal arteries distally to or toward the iliac arteries.

In treating an aneurysm with a stent-graft, the stent-graft typically is placed so that one end of the stent-graft is situated proximally or upstream of the diseased portion of the vessel and the other end of the stent-graft is situated distally or downstream of the diseased portion of the vessel. In this manner, the stent-graft extends through the aneurysmal sac and beyond the proximal and distal ends thereof to replace or bypass the weakened portion. The graft material typically forms a blood impervious lumen to facilitate endovascular exclusion of the aneurysm.

Such prostheses can be implanted in an open surgical procedure or with a minimally invasive endovascular approach. Minimally invasive endovascular stent-graft use is preferred by many physicians over traditional open surgery techniques where the diseased vessel is surgically opened and a graft is sutured into position such that it bypasses the aneurysm. The endovascular approach, which has been used to deliver stents, grafts, and stent grafts, generally involves cutting through the skin to access a lumen of the vasculature. Alternatively, lumenar or vascular access may be achieved percutaneously via successive dilation at a less traumatic entry point. Once access is achieved, the stent-graft can be routed through the vasculature to the target site. For example, a stent-graft delivery catheter loaded with a stent-graft can be percutaneously introduced into the vasculature (e.g., into a femoral artery) and the stent-graft delivered endovascularly across the aneurysm where it is deployed.

When using a balloon expandable stent-graft, balloon catheters generally are used to expand the stent-graft after it is positioned at the target site. When, however, a self-expanding stent-graft is used, the stent-graft generally is radially compressed or folded and placed at the distal end of a sheath or delivery catheter. Upon retraction or removal of the sheath or catheter at the target site, the stent-graft self-expands.

More specifically, a delivery catheter having coaxial inner and outer tubes arranged for relative axial movement therebetween can be used and loaded with a compressed self-expanding stent-graft. The stent-graft is positioned within the distal end of the outer tube (sheath) and in front of a stop fixed to the inner tube. Once the catheter is positioned for deployment of the stent-graft at the target site, the inner tube is held stationary and the outer tube (sheath) withdrawn so that the stent-graft is gradually exposed and allowed to expand. The inner tube or plunger prevents the stent-graft from moving back as the outer tube or sheath is withdrawn. An exemplary stent-graft delivery system is described in U.S. Patent Application Publication No. 2004/0093063, which published on May 13, 2004 to Wright et al. and is entitled Controlled Deployment Delivery System, the disclosure of which is hereby incorporated herein in its entirety by reference.

Regarding proximal and distal positions referenced herein, the proximal end of a prosthesis (e.g., stent-graft) is the end closest to the heart (by way of blood flow) whereas the distal end is the end furthest away from the heart during deployment. In contrast, the distal end of a catheter is usually identified as the end that is farthest from the operator, while the proximal end of the catheter is the end nearest the operator.

Although the endoluminal approach is much less invasive, and usually requires less recovery time and involves less risk of complication as compared to open surgery, among the challenges with this approach are fixation of the prosthesis and prosthesis migration. For example, the outward spring force of a self-expanding stent-graft may not be sufficient to prevent migration. This problem can be exacerbated when the vessel's fixation zone significantly deviates from being circular. And when there is a short landing zone, for example, between an aortic aneurysm and a proximal branching artery (e.g., one of the renal arteries, or the carotid or brachiocephalic artery), small deviations in sizing or placement may result in migration and or leakage.

Current endovascular devices incorporate stent-graft over-sizing to generate radial force for fixation and/or sealing and some have included fixation mechanisms comprising radially extending members such as tines, barbs, hooks and the like that engage the vessel wall to reduce the chance of migration. In some abdominal aortic aneurysm applications, a suprarenal stent and hooks are used to anchor the stent-grafts to the aorta. However, abdominal aortic aneurysm stent-grafts typically require an anchor or landing zone of about 10-15 mm to achieve the desired fixation and seal efficacy. In some cases, such an anchoring or landing zone does not exist due to diseased vasculature or challenging anatomy. Other attempts to improve fixation and/or sealing between the prosthesis and an endoluminal wall have included using adhesives and growth factor. There remains a need to develop and/or improve seal and/or fixation approaches for endolumenal or endovascular prostheses placement.

### SUMMARY OF THE INVENTION

The present invention involves improvements in prosthesis fixation and overcomes disadvantages of prior art.

In one embodiment according to the invention, a method of securing a tubular prosthesis to an inner wall of a passageway defining a lumen in a human body comprises endoluminally advancing a tubular prosthesis to a site in in a human body; endoluminally advancing a plurality of fasteners to a plurality of sites within the prosthesis; and passing the fasteners from an inner surface of the prosthesis through the prosthesis and a wall of the passageway. In another embodiment according to the invention, a method of securing a tubular prosthesis to an inner wall of a vessel in a human patient comprises endoluminally advancing a tubular prosthesis having an inner surface through a vessel in a human patient to a region of the vessel; endoluminally advancing a plurality of fastener carriers, each carrying at least one fastener, through the vessel to the region; and deploying the fasteners from the carriers and passing the fasteners from the inner surface of the prosthesis through the prosthesis and the vessel to secure the prosthesis to the vessel.

In another embodiment according to the invention, a prosthesis delivery system comprises a tubular prosthesis having an inner wall; a plurality of guide members extending from the inner wall; and a plurality of fasteners coupled to one or more of the guide members.

In another embodiment according to the invention, a prosthesis delivery system comprises a catheter having a lumen; a tubular prosthesis having an inner wall surface and being disposed in the catheter lumen; a plurality of guide members extending from the inner wall surface; and a plurality of fasteners coupled to one or more of the guide members.

In another embodiment according to the invention, endovascular fastener delivery apparatus comprises a catheter having a proximal end and a distal end; at least one fastener delivery tube disposed in the catheter and having a proximal end portion and a distal end portion; at least one self-closing fastener disposed in the fastener delivery tube; and an expander including a radially extendable arm pivotally coupled to said distal end portion of said at least one fastener delivery tube.

In another embodiment according to the invention, a graft implantation device comprises a catheter having a distal end and a proximal end, the distal end including a tubular graft for implantation, a fastener delivery mechanism for delivering at least two fasteners simultaneously and being disposed within said tubular graft, wherein in a delivery configuration of the catheter, tubular graft, and fastener delivery mechanism have a delivery outside diameter to provide a profile adapted for delivery through the vasculature to a treatment site; wherein in a pre-deployment configuration of the catheter, tubular graft, and fastener delivery mechanism, said tubular graft is held by said fastener delivery mechanism extended radially to a larger diameter than the delivery outside diameter and against a surrounding tissue wall, where upon actuation the fastener delivery mechanism for delivering at least two fasteners simultaneously delivers the at least two fasteners through the tubular graft and into a surrounding tissue, whereby the at least two fasteners fix the tubular graft to the surrounding tissue.

In another embodiment according to the invention, graft implantation apparatus comprises a catheter having a distal end portion and a proximal end portion, the catheter being sized for delivery through vasculature of a human patient; a plurality of fasteners; a plurality of fastener delivery carriers disposed in the catheter, each fastener delivery carrier having a distal end portion, and each fastener delivery carrier carrying at least one of the fasteners; an expander coupled to the carriers to radially expand the carrier distal end portions; and a tubular graft surrounding at least a portion of the distal end portion of the delivery carriers and being disposed in the distal end portion of the catheter.

The above is a brief description of some deficiencies in the prior art and advantages of embodiments according to the present invention. Other features, advantages, and embodiments according to the present invention will be apparent to those skilled in the art from the following description and accompanying drawings, wherein, for purposes of illustration only, specific embodiments are set forth in detail.

### BRIEF DESCRIPTION OF THE DRAWINGS

FIG. 1 diagrammatically illustrates endovascular prosthesis delivery apparatus according to one embodiment of the invention.

FIG. 2 is an enlarged view of a distal end portion of the apparatus of FIG. 1.

FIG. 3A diagrammatically illustrates one embodiment of fastener delivery apparatus according to the invention in a pre-deployment state.

FIG. 3B diagrammatically illustrates the apparatus of FIG. 3A with the distal ends of the fastener tubes positioned at ejection sites and multiple fasteners deployed simultaneously.

FIG. 3C is a partial sectional view of FIG. 3A taken along line 3C-3C.

FIG. 3D is a partial sectional view of FIG. 3A taken along line 3D-3D without plunger 120 for purposes of clarity.

FIG. 3E is a partial sectional view of FIG. 3B taken along line 3E-3E without plunger 120 for purposes of clarity.

FIG. 3F is an enlarged view of the section encircled with line 3F in FIG. 3B.

FIGS. 4A and 4B illustrate one embodiment of a fastener plunger according to the invention where 4A shows the plunger is in a pre-deployment state and FIG. 4B shows the plunger being actuated and a plurality of fasteners being deployed.

FIG. 4C diagrammatically illustrates the embodiment of FIG. 4A with a tubular graft surrounding a distal end portions of the fastener tubes and disposed in a delivery catheter.

FIG. 4D diagrammatically illustrates the tubular graft of FIG. 4C after it has been pushed out of the distal end of the catheter, radially expanded and fasteners passed therethrough.

FIG. 4E illustrates one embodiment of a fastener according to the invention.

FIG. 4F illustrates the fastener of FIG. 4D restrained in an open position in a fastener delivery tube.

FIGS. 5A-5D diagrammatically illustrate guidance of a fastener delivery tube to a target site on a prosthesis and deployment of a fastener where FIG. 5A shows the fastener delivery tube being tracked along a guide member to a target site, FIG. 5B shows a the fastener delivery tube at the target site and a fastener being deployed therefrom, FIG. 5C shows the fastener fully deployed and the fastener delivery tube removed, and FIG. 5D shows the guide member removed from the prosthesis.

FIG. 6 illustrates another embodiment of guide apparatus according to the invention.

FIG. 7A is a partial sectional view of another embodiment of guide apparatus according to the invention.

FIG. 7B is end view of the guide apparatus of FIG. 7A taken along line 7B-7B.

FIG. 8 is a partial sectional view of another embodiment of fastener delivery apparatus according to the invention.

FIGS. 9A-9C diagrammatically illustrate one method of endoluminally delivering fasteners serially fasteners using the apparatus of FIG. 8, where FIG. 9A shows the fastener delivery tube of FIG. 8 positioned in the prosthesis, FIG. 9B shows four guide members provided slack and one tightened to move the distal, deployment end of the fastener delivery tube to a target site, and FIG. 9C shows a different set of four guide members provided slack and a different guide member tightened to move the distal, deployment end of the fastener delivery tube to another target site.

FIG. 10A is a partial sectional view of another embodiment of fastener delivery apparatus according to the invention.

FIG. 10B illustrates the longitudinal slot in the fastener tube expander tube of FIG. 10A.

FIG. 10C is an end view of the expander tube of FIG. 10B.

FIGS. 11A and 11B diagrammatically illustrate a pre-deployment state FIG. 11A and deployment state FIG. 11B using the apparatus of FIG. 8.

FIGS. 12A and 12B illustrate a method of using any of the apparatus described herein for endoluminally deploying fasteners to secure a prosthesis such as a graft or stent-graft to the proximal landing of an abdominal aortic aneurysm.

### DETAILED DESCRIPTION

The following description will be made with reference to the drawings where when referring to the various figures, it should be understood that like numerals or characters indicate like elements. When referring to catheters, delivery devices and loaded fasteners described below the proximal end is the end nearest the operator and the distal end is farthest from the operator.

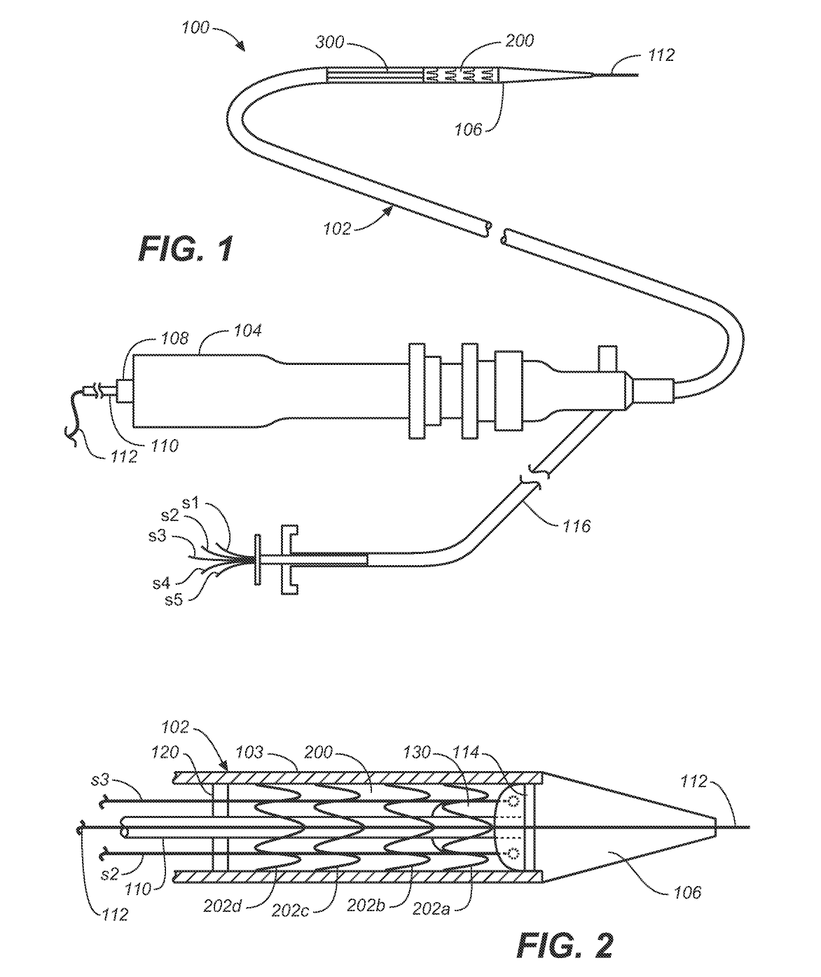

Referring to FIG. 1, one embodiment of a prosthesis delivery system according to the invention is shown and generally designated with reference numeral 100. Prosthesis delivery system 100 comprises catheter 102, which includes catheter sheath 103, control handle 104, flexible tapered tip member (or obturator 106), which can form a portion of the distal end of the catheter. In the embodiment illustrated in FIG. 1, system 100 is equipped with prosthesis 200 and fastener delivery apparatus 300.

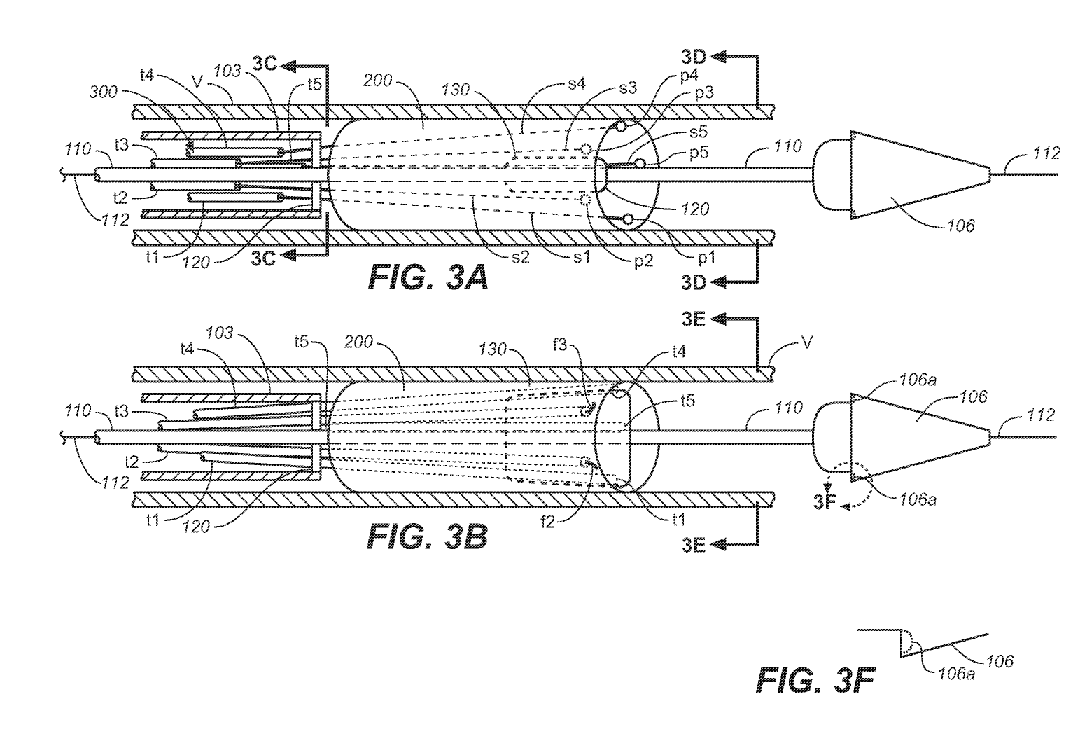

Handle 104 includes an inlet 108, through which central guidewire lumen 110 enters the handle and extends to flexible tapered tip member 106, which has an axial bore for slidably receiving guidewire 112. Tapered tip member 106 is placed at the distal end of catheter sheath 103 and handle 104 is affixed to the proximal end of catheter sheath 103 in the vicinity of access tube 116, which is coupled to handle 104 and in fluid communication with catheter sheath 103, which has a size of about 12 to 28 French. A guidewire 112 can be slidably disposed in guidewire lumen 110 and catheter 102 tracked thereover. When the prosthesis to be delivered is a self-expanding graft or stent-graft (such as stent-graft 200), it generally is radially compressed or folded and placed in the distal end portion of the delivery catheter and allowed to expand upon deployment from the catheter at the target site as will be described in detail below. Stent-graft 200 can include a plurality of undulating stent elements to support the tubular graft material as is known in the art. Although the stent framework is shown with a particular configuration in FIG. 2, it should be understood that that configuration is merely provided for exemplary purposes and other configurations can be used. Further, the stent framework can be nitinol or any other suitable material. The graft material for any of the prostheses described herein also can be any suitable material such as Dacron® or expanded polytetrafluoroethylene (ePTFE). A graft by itself without a framework may also be used.

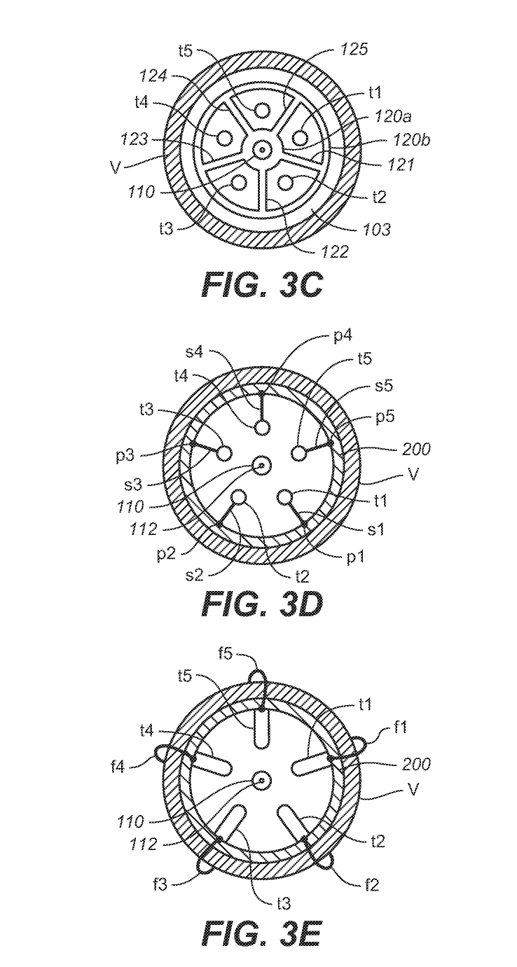

Referring to FIG. 2, one delivery catheter system configuration according to the invention is shown in a pre-deployment loaded state. Sheath 103 (outer tube) and guidewire tube 110 (inner tube) are coaxial and arranged for relative axial movement therebetween. The prosthesis (e.g., stent-graft 200) is positioned within the distal end of outer tube (sheath) 103 and in front of plunger or stop 120, which is concentric with and secured to inner guidewire tube 110. In the illustrative embodiment, pusher member or stop 120 has a wagon wheel shaped configuration with a hub 120a having a central access bore, which provides access for guidewire tube 110, and spokes 121, 122, 123, 124, and 125, which radially extend from hub 120a to the outer circular portion 120b of stop 120 and form spaces therebetween to allow fastener tubes t1, t2, t3, t4, and t5 to slidably pass therethrough as shown in FIG. 3C. Once the catheter is positioned for deployment of the prosthesis at the desired site, the plunger is held stationary and the outer tube or sheath withdrawn so that the prosthesis, e.g., stent-graft, is gradually exposed and allowed to expand.

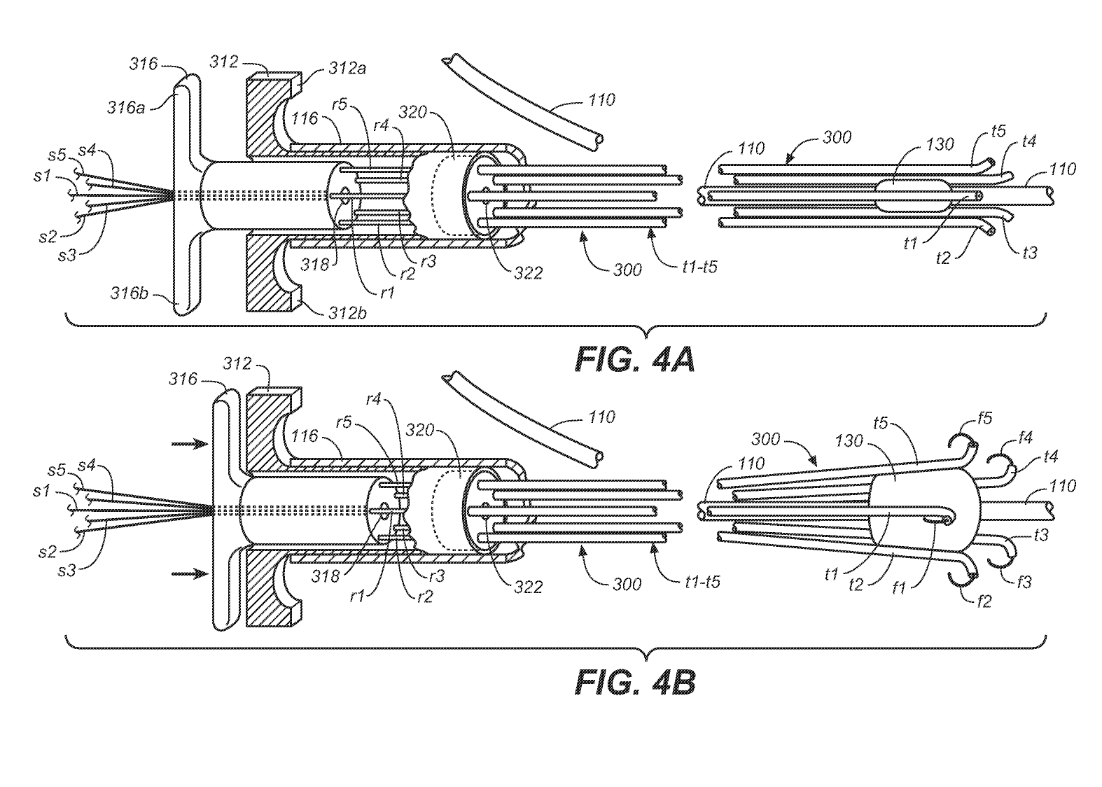

Fastener tubes t1, t2 . . . tn have a length of at least about the length of the delivery catheter, which ranges from about 30-90 cm depending on the application plus a margin of about 20-45 cm to accommodate exiting handle 104 and access tube 116. Accordingly, the fastener tube length is at least about 50 cm and can be up to about 135 cm. Although one configuration for allowing passage of the fastener tubes is shown, it should be understood that other configurations can be used. In one variation, stop 120 is not included and the distal ends of the fastener tubes provide the mechanism to push stent-graft 200 distally to deploy the prosthesis. In another variation illustrated in FIGS. 4C and 4D, which will be described in more detail below, a tubular graft 250 without a stent framework surrounds the fastener tubes, which push the tubular graft out from the catheter without stop 120 and radially expand the tubular graft. However, stop 120 can be incorporated in that embodiment according to yet a further variation.

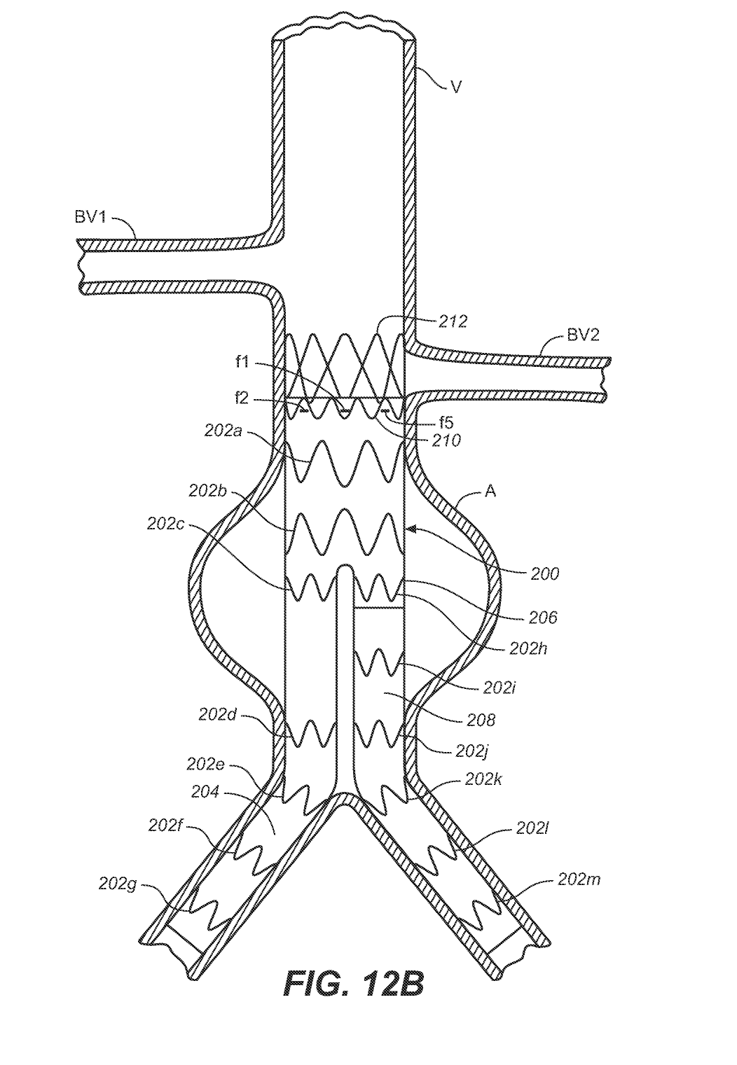

In the example where prosthesis 200 comprises a stent-graft as shown in the illustrative embodiment depicted in FIG. 2, the stent-graft comprises a tubular graft member and a plurality of annular undulated stent elements, such as stent elements 202a,b,c,d to provide structural support to the graft as is known in the art. As shown in FIG. 12B, an undulating bare spring element 212 also can be sutured or otherwise attached to the proximal end of the prosthesis and/or an annular undulating wire 210 having an undulating configuration secured to the proximal end of the prosthesis to provide radial strength as well. The spring has a radially outward bias so that when it is released from a radially restrained state it expands outwardly to secure the proximal portion of the prosthesis to the target passageway wall. Another undulating wire 210 can be attached to the prosthesis distal end as well or in the alternative. More specifically, a support spring 210 can be provided at one or both ends of the prosthesis. The stent and support elements can be positioned on the interior and/or exterior of the graft member and secured thereto by suturing or other conventional means.

Returning to FIG. 2, a radiopaque ring 114 can be provided on the inside of the distal end portion of sheath 103 in overlapping relation to tapered tip 106 to assist with imaging the distal end of sheath 103 using fluoroscopic techniques. Alternatively, radiopaque ring 114 can be provided on the proximal end of the tapered tip.

A plurality of guide members s1, s2, s3, s4, s5 . . . sn, which can be in the form of a flexible elongated member such as a suture, a wire, thread, or filament, each have one end attached to the inner surface of the graft material of stent-graft 200 or a portion of the stent structure at an attachment point or fastener target site p1, p2, p3, p4, p5 . . . pn. Each guide member extends to a fastener guide tube t1, t2, t3, t4, t5 . . . tn and is slidably coupled to a respective fastener tube so that the guide tube can be tracked thereover. The guide members and fastener tubes extend through catheter 102 between catheter sheath 103 and guidewire lumen 110 out from catheter 102 and into access tube lumen 116, which extends from the distal end potion of handle 104 and is in fluid communication with the proximal end of catheter sheath 103 which terminates at the distal end portion of handle 104. The fastener tubes and guide members are coupled to actuator or plunger 310 as will be described in more detail below with reference to FIGS. 4A and 4B. Although five guide members and corresponding fastener tubes, each loaded with a single fastener, is shown, more or fewer guide members and fastener tubes can be used and each fastener tube can be loaded with more than one fastener. Further, although the attachment points are shown in an annular, equidistantly spaced arrangement, other configurations or arrangements can be used including, but not limited to, spiral arrangements and arrangements where the attachment points are not equidistantly spaced.

Referring to FIGS. 3A-3D, diagrammatic views illustrating prosthesis deployment and fixation according to one embodiment of the invention will be described. Once the catheter is positioned for deployment of the prosthesis at the desired site, the inner member or guidewire lumen 110 with stop 120 are held stationary and the outer tube or sheath 103 withdrawn so that tapered tip 106 is displaced from sheath 103 and the stent-graft gradually exposed and allowed to expand. Stop 120 therefore is sized to engage the distal end of the stent-graft as the stent-graft is deployed. The proximal end portions of the sheath 103 and inner tube or guidewire lumen 112 are coupled to and manipulated by handle 104. Tapered tip 106 optionally can be configured with an annular recess or cavity 106a formed in its distal end portion and configured to receive and retain the leading or proximal end portion of the prosthesis in a radially compressed configuration before allowing its expansion during deployment (FIG. 3F). Alternatively, any of the stent-graft deployment systems described in U.S. Patent Application Publication No. 2004/0093063, which published on May 13, 2004 to Wright et al. and is entitled Controlled Deployment Delivery System, the disclosure of which is hereby incorporated herein by reference in its entirety, can be incorporated into stent-graft delivery system 100.

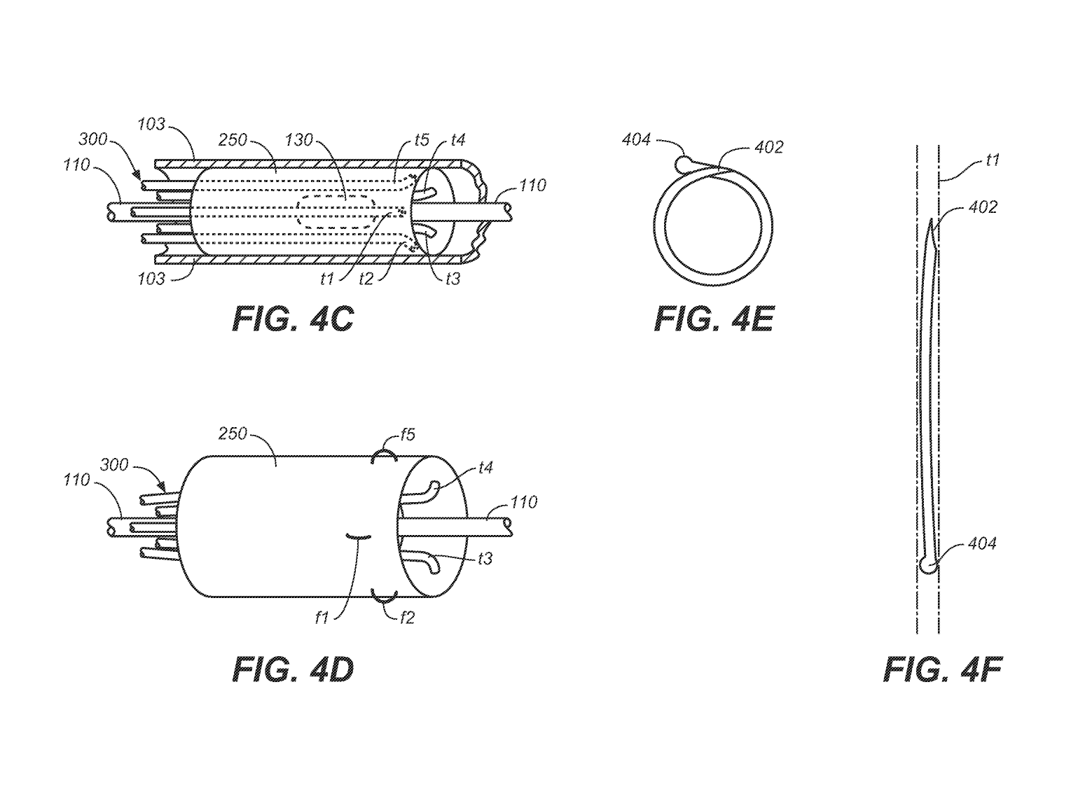

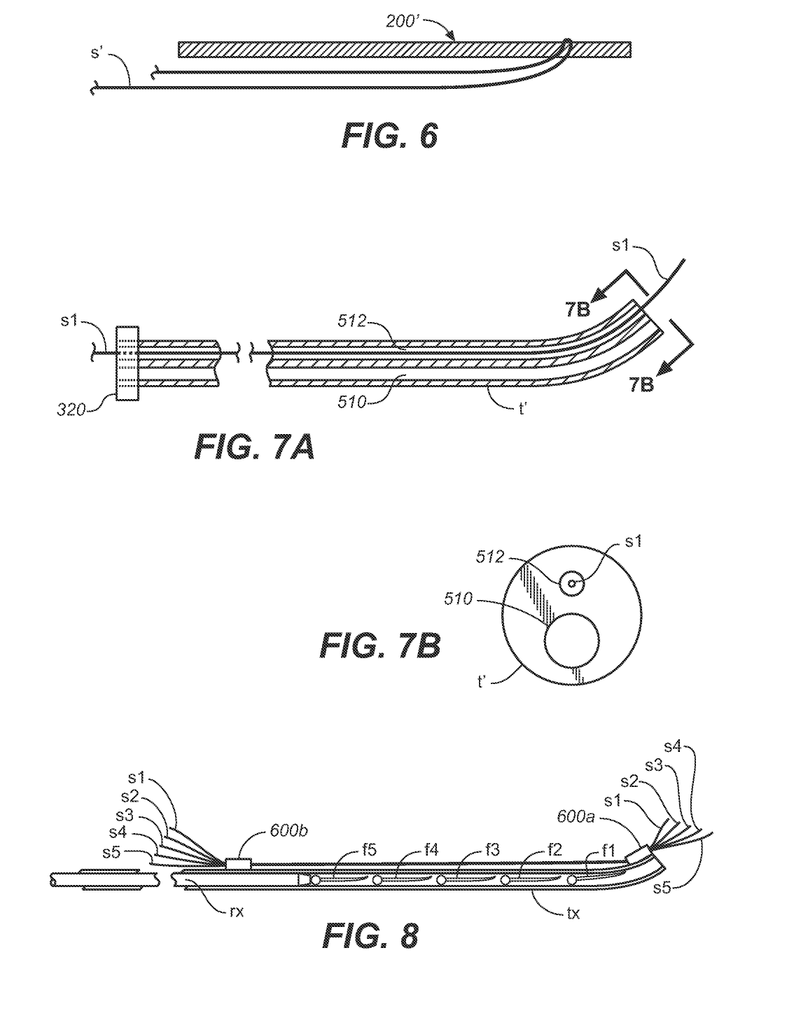

Referring to FIG. 3A, the prosthesis is deployed and radially expanded. Fastener tubes f1-f5 are then advanced and guided along guide members s1-s5 to attachment points or fastener target sites p1-p5 as shown in FIG. 3B. Examples, of fastener tube guide member receiving mechanisms that track over a respective guide member will be described in more detail below in connection with FIGS. 5A, 5B and 7. Returning to FIG. 3B, once the distal ends of fastener tubes f1-f5 are positioned at or against target sites p1-p5, fasteners (see e.g., fasteners f2 and f3 in FIG. 3B) are deployed from the fastener tubes to secure the prosthesis to the vessel wall. The operator can tactilely determine when the distal ends of the fastener tubes are in contact with the inner wall of the prosthesis. Alternatively, a radiopaque marker can be provided on each guide member at the attachment point and on the distal end of each fastener delivery tube so that the relative positions of the distal end of each fastener delivery tube and corresponding guide member attachment point can be fluoroscopically monitored. In one variation, each fastener tube marker can be placed at a predetermined distance from the distal end of the tube. That information would be processed with traditional means to monitor the relative positions of each guide member attachment site and fastener delivery tube distal end fluoroscopically.

The distal end portions of each fastener delivery tube also can be bent radially outward as shown, for example, in FIG. 4A, so that when the fastener tube end is urged against the inner wall of the prosthesis, the fastener is pointed toward the inner wall of the prosthesis to enhance fastener penetration. Typically, the distal end of each fastener tube will be configured or bent so that the fastener can penetrate the prosthesis along a line that is within about 60 degrees of a line that extends radially outward from the longitudinal axis of the guidewire tube toward the designated attachment point for the fastener.

The fasteners can be self-closing fasteners having a loop shaped memory set closed configuration. They can be made from nitinol wire and placed in the desired shape (e.g., that shown in FIG. 4E) and heated for about 5-15 minutes in a hot salt bath or sand having a temperature of about 480-515° C. They can then be air cooled or placed in an oil bath or water quenched depending on the desired properties. In one alternative, the fasteners can be surgical grade stainless steel that is deformed to assume such a preshaped configuration. In a further embodiment, the fasteners can be polymeric material with a preshaped loop configuration to which they return when released from the fastener tube.

Returning to FIGS. 3A and 3B, optional expandable balloon of conventional construction can be used to provide radial support for the distal end portions of the fastener tubes to stabilize the tubes and/or minimize or eliminate fastener tube movement away from the target sites during fastener deployment. In the illustrative example, the expandable balloon is secured to a portion of guidewire tube 110 and spaced from tapered tip member 106 such that when the guidewire tube and tapered tip are advanced to fully deploy the stent-graft as shown in FIG. 3A, the uninflated balloon is radially aligned with the fastener target sites. An end view of stop 120 is shown in FIG. 3C, which is a view taken along line 3C-3C in FIG. 3A. After the fastener tubes are advanced so that their distal ends are close to or abut the inner wall of prosthesis 200, the balloon is inflated so that it expands and urges the fastener tubes against the inner wall of the prosthesis to stabilize the fastener tubes in a desired position as shown in FIG. 3B. Once the tubes are stabilized, the fasteners are deployed. The balloon can be polyurethane and fluidly coupled to an external pressure source through a lumen formed in the wall of the guidewire tube as would be apparent to one of skill in the art.

FIG. 3D diagrammatically shows a sectional view taken along line 3D-3D in FIG. 3A where the fastener tubes f1-f5 are ready to be tracked along guide s1-s5 to target sites or points p1-p5. FIG. 3E diagrammatically illustrates fastener tubes t1-t5 tracked to the target sites p1-p5 and positioned against the inner wall of prosthesis 200, which, in its expanded state, forms contact with the inner wall of vessel V. A plurality of fasteners f1-f5 is shown deployed to fix the prosthesis to the vessel and enhance seal formation therebetween. Typically, at least about a 15mm landing zone is required to secure a stent-graft or graft to a vessel. However, the fastener procedure described herein and illustrated throughout the figures, can facilitate the requisite fixation and sealing when the landing zone is only about 5 mm in length. The region of a vessel between the aneurysm and the nearest branch vessel is referred to as the landing zone. One such landing zone is depicted in FIGS. 5A-D and designated with reference character LZ.

Referring to the embodiment illustrated in FIGS. 4A and 4B, ejector apparatus to eject fasteners f1-f5 from fastener tubes f1-f5 is shown integrated into fastener delivery apparatus 300. In the illustrative embodiment, the ejector apparatus comprises a plurality of pusher rods r1-r5 each of which is slidably disposed in a fastener tube and configured to push a fastener out from the tube. The proximal end portions of the pusher rods are fixedly secured to plunger 316 (e.g., to the distal portion of the plunger), which can include radial extending and laterally spaced grip members or wings 316a and 316b. Plunger 316 also includes a central bore 318 though which guide members s1-s5 pass.

Plunger 316 is slidably disposed in tubular fastener tube holding member 312, which is slidably disposed in access tube 116 and which can include radially extending and laterally spaced grip members or wings 312a and 312b. The proximal ends of fastener tubes f1-f5 are fixedly secured in through holes that extend through plug or disk member 320, which is secured to the inner wall of tubular fastener tube holding member 312. In this manner, tube holding member 312 can be advanced or retracted to advance or retract tubular members t1-t5. Plug or disk member 320 can include a through bore 322 through which guide members s1-s5 can be passed. When plunger 316 is actuated, the pusher rods r1-r5 are simultaneously advanced in fastener tubes s1-s5 to eject all of the fasteners (e.g., fasteners f1-f5) from the fastener tubes. In this manner all of the fasteners can be ejected in a single thrust of plunger 316. Further, the pusher member and fastener tube lengths can be selected so that all of the fasteners are simultaneously ejected.

Referring to FIGS. 4C and 4D the ejector apparatus of FIG. 4A is shown in one variation where tubular graft 250, which does not include a stent framework, surrounds the fastener tubes. In FIG. 4C, graft 250 and distal portions of the fastener tubes are shown disposed within the distal end portion of delivery catheter tube 103. Graft 250 is arranged about the fastener tubes so that the fastener tubes carry graft 250 with them as they are pushed out from catheter tube (sheath) 103. In one embodiment, tubular graft extends over the curved distal ends of the fastener tubes and that relationship with the compaction of the graft about the fastener tubes allows the tubes to carry graft 250 when they are advanced. When graft 250 is outside catheter sheath 103, the balloon can be expanded to radially expand the fastener tubes and tubular graft 250 as shown in FIG, 4D, where the fasteners also are shown deployed. In use, the graft would be positioned at the desired endoluminal site before radial expansion and fastener deployment. In one variation, stop 120 can be incorporated as described above to assist in pushing graft 250 out from catheter sheath 103.

Referring to FIG. 4E, one fastener embodiment is shown and designated with reference numeral 400. Fastener 400 includes a sharp piercing end 402 and an enlarged end portion 404 that the distal end of a respective pusher rod pushes through a fastener tube. The fastener can be provided with a memory shaped closed loop configuration as described above. FIG. 4F shows one fastener 400 restrained in an open configuration in fastener tube t1. Such a fastener can be loaded in all of the delivery tubes in a similar manner.

Referring to FIGS. 5A-5D, a coupling system for slidably coupling the fastener delivery tubes to guide members is diagrammatically shown. Although a plurality of fastener tubes are shown in FIG. 4A, a single tube is provided in this example for simplification. The coupling system generally comprises one or more tubes or sleeves that extend from or are attached to a respective fastener tube and are configured to allow a guide member to slidably pass therethrough. In this example, fastener delivery tube t1 has one tubular member 500a extending from or attached to its outer surface at its distal end and another tubular member 500b extending from or attached to an intermediate portion of its outer surface. Fastener delivery tube t1 is sized to slidably receive one of the guide members such as guide member s1 so that fastener delivery tube t1 can be tracked along the guide member (FIG. 5A) to a target site where the guide member is attached to the inner wall of prosthesis 200 as shown in FIG. 5B. The other fastener delivery tubes are similarly sized and provided with similar coupling systems. Although not shown, it should be understood that one or more additional tubular members can be provided along tubular member t1. After fastener tube t1 is positioned at the target site, pusher rod r1 is advanced to deploy fastener f1 (FIG. 5B). Fastener tube t1 is then retracted (FIG. 5C) and the guide member cut and removed from graft 200 (FIG. 5D) using traditional endoscopic techniques.

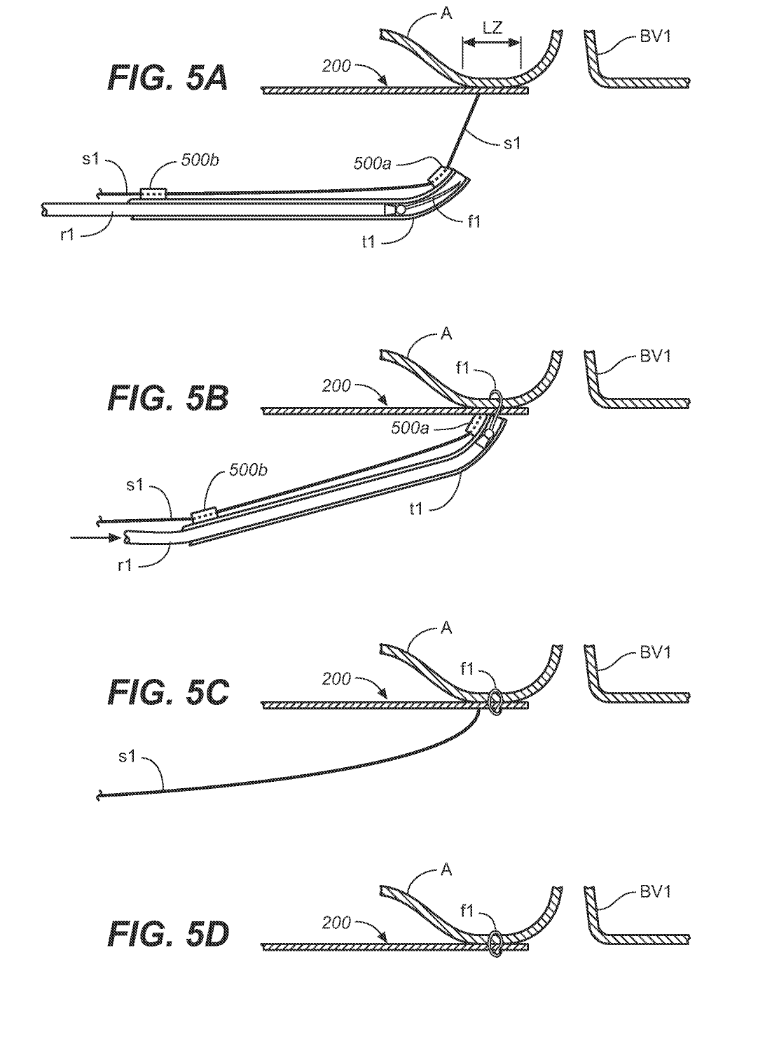

Referring to FIG. 6, another guide member arrangement is shown where the guide member “s′” is looped through prosthesis 200′, which can be, for example, a graft or stent-graft. The guide member enters and exits the inner wall of the prosthesis. Both ends of the guide member extend through bore 322 and exit plunger 316 (see FIGS. 4A & 4B). In this manner, one end of the guide member can be pulled to remove the guide member from the vessel after the prosthesis has been secured in place.

In the embodiment illustrated in FIG. 7A, another mechanism for slidably coupling a fastener tube to a guide member is shown. In this embodiment, fastener delivery tube “t′” includes a first lumen 510 that is sized for passage of a fastener and pusher rod thererthough and a second lumen 512 that is sized so that a guide member can slidably pass therethrough. A guide member such as guide member s1 passes through lumen 512 and disk 320 and extends out from plunger 316.

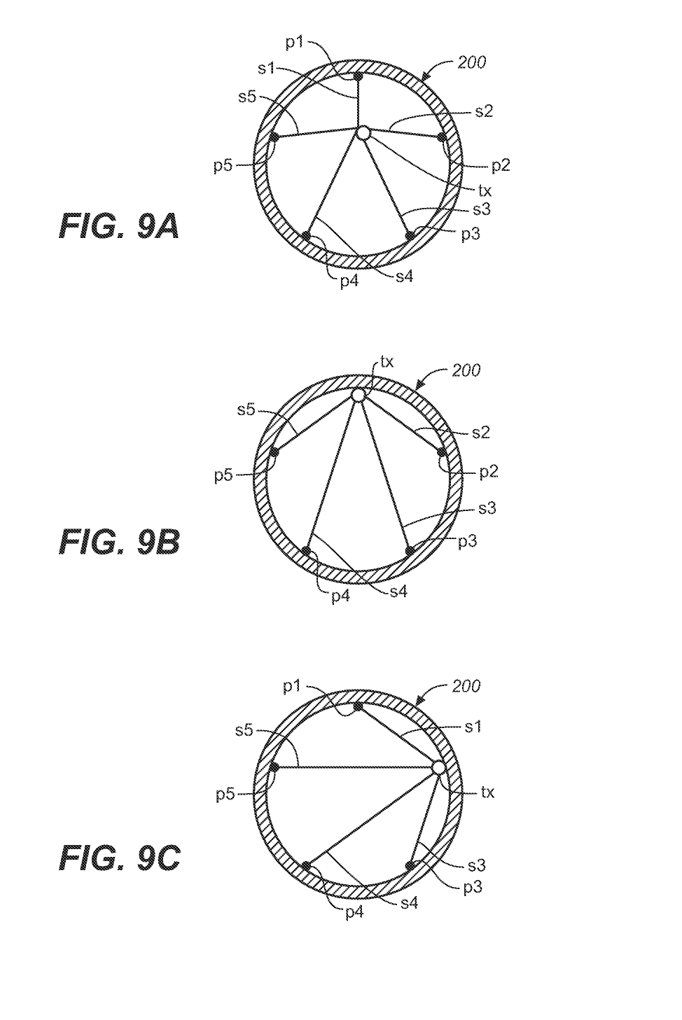

Referring to FIG. 8, an alternative fastener delivery apparatus including a single fastener delivery tube “tx” loaded with a plurality of serially aligned fasteners is shown. Although five fasteners, fasteners f1-f5, are shown serially loaded in the fastener tube lumen in front of the distal end of pusher rod “rx,” fastener delivery tube “tx” can be loaded with more or fewer fasteners. A plurality of guide members, e.g., guide members s1-s5, are slidably coupled to fastener delivery tube “tx” through one or more couplings such as sleeve or tubular members 600a and 600b, which extend from or are attached to the fastener tube in the same manner as described above regarding sleeves 500a and 500b with sleeve 600a being at the distal end of fastener delivery tube “tx”. Fastener tube “tx,” pusher rod “rx,” and the guide members extend back through catheter sheath 103 and exit the system through access tube 116 where the operator can manipulate these elements to position and deploy the fasteners at the target sites. The fastener delivery tube “tx” can be coupled to disk 320 and the guide member passed through bore 322 of disk 320 (see FIGS. 4A & 4B) or tube holding member 312 eliminated and the fastener delivery tube and guide member simply passed through plunger 316. In a further arrangement, plunger 316 also can be eliminated. The fasteners also can vary from the fasteners shown in FIG. 8. For example, the rounded end portions can be flat, dimpled, or otherwise configured to enhance their cooperation with a trailing fastener that pushes it forward.

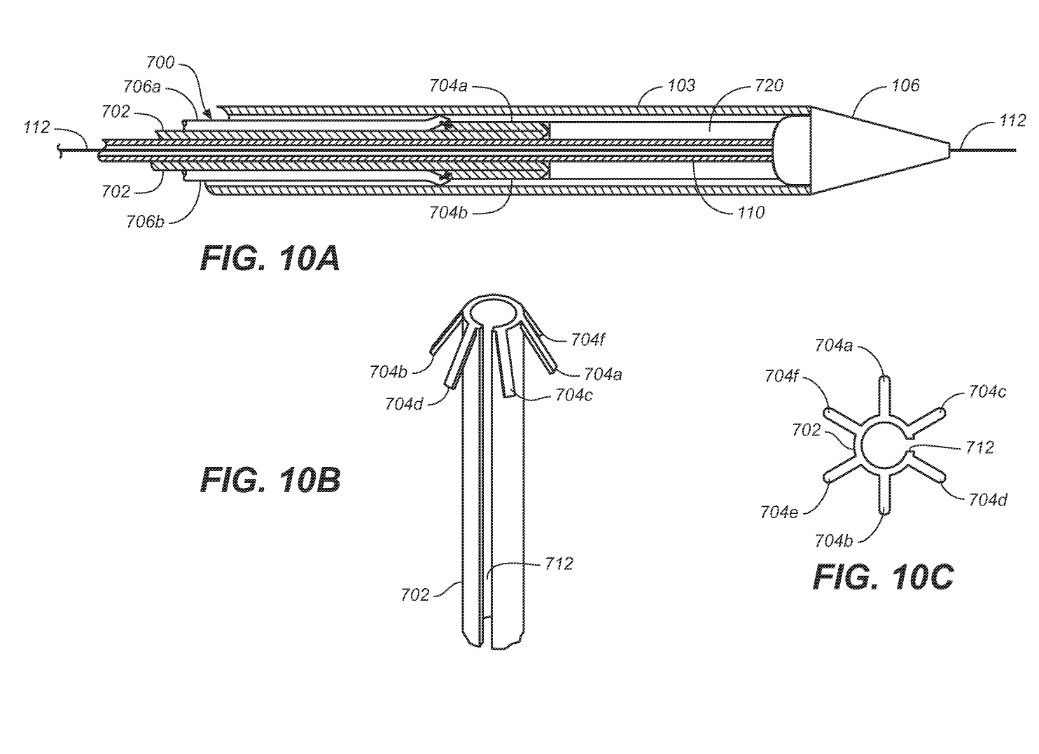

One method of using fastener delivery tube “tx” is diagrammatically shown in FIGS. 9A-C. The proximal end of fastener tube “tx” is manipulated (e.g., pushed) to position its distal end in the vicinity of target sites p1-p5. All of the guide members are provided slack (FIG. 9A) and then the slack is taken up in one guide member (guide member s1 in FIG. 9B) and fastener tube “tx” advanced. As the fastener tube is advanced, it will track along the taut guide member so that its distal end can be positioned close to or in contact with the inner wall of prosthesis 200. Pusher rod “rx” can then be pushed to deploy a single fastener. After the fastener is fully deployed to secure a portion of the prosthesis to a vessel wall, guide member s1 is given slack, the slack in guide member s2 taken up, and fastener delivery tube “tx” advanced. In this manner, the distal end of fastener tube tx is positioned close or in contact with a second target site (FIG. 9C). The fastener tube can be slightly retracted before tightened guide member s2 and then advanced after the slack in guide member s2 is taken up. The apparatus also allows the operator to move sequentially from p1 to p2 to p3 to p4 to p5 or in any other sequence. In this manner, the distal end of the fastener tube can be repositioned at the various target sites and a fastener at each site.

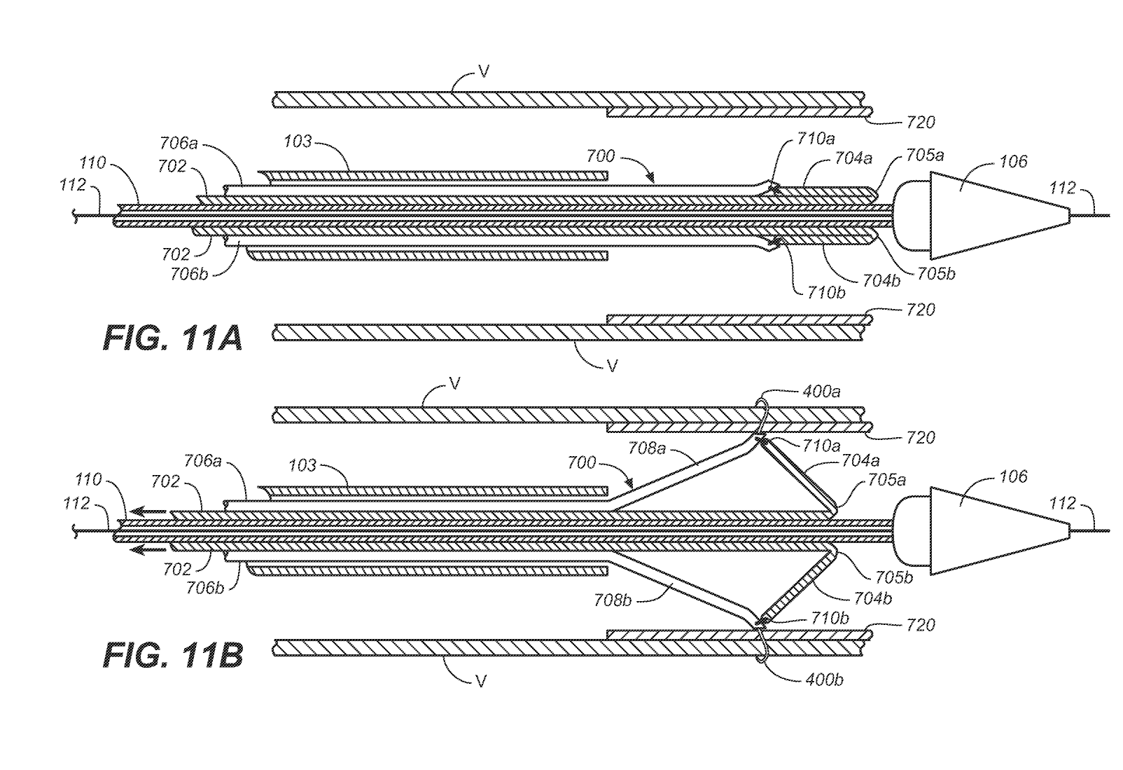

Referring to FIGS. 10A-C and 11A-11B another fastener delivery apparatus for incorporation in prosthesis delivery system 100 is shown and generally designated with reference numeral 700. Fastener delivery apparatus 700 includes a tubular member 702 that tracks over guidewire tube 110 and one or more fastener tubes 706a, b . . . n, which are coupled to tubular member 702. Referring to FIGS. 10B and 10C, tubular member 702 can include a slot 712 extending its entire length with the slot having a width slightly greater than the diameter of guidewire lumen 110 to allow tube 702 to branch away from guidewire lumen 110 and enter access tube 116 with fastener delivery tubes 706a, 706b . . . 706n.

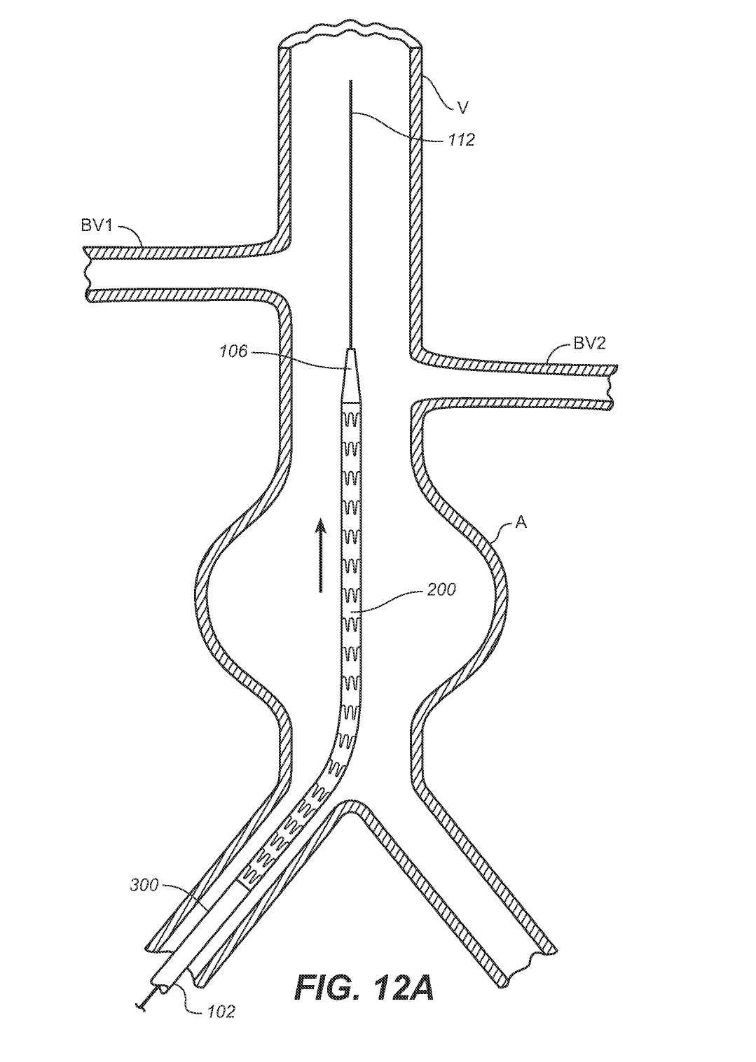

In the embodiment illustrated in FIG. 10A-C, a plurality of struts 704a,b . . . n extend from or are attached to the distal end portion of tubular member 702 and form an expandable member. Although six struts are shown equidistantly spaced in FIGS. 10B and 10C, other arrangements and numbers of struts can be used. Typically two to eight struts are used. Struts 704a,b . . . n are spaced from one another about the circumference of the distal end portion of tubular member 702 and extend from tube 702 through flexible portions or hinges 705a,b . . . n so that they can pivot or move to the position shown in FIG. 11B where they radially extend from tube 702. The distal end portions of struts 704a,b . . . n are pivotally coupled to respective fastener tubes 706a,b . . . n through flexible portions or hinges 710a,b . . . n so that the struts and fastener tube pairs can pivot relative to one another (see FIGS. 11A & B). In one example, foregoing hinges can comprise a flexible connection between the members being coupled. In another example, the portion of material at the juncture of tube 702 and a strut can have a thinner wall thickness than that of the tube and strut to provide a hinge. Each of the fastener tubes can be provided with a fastener and pusher rod as shown in FIG. 4A to facilitate fastener deployment at the target site. When using the fastener deployment apparatus of FIG. 4A, a central bore would be provided in disk 320 in place of bore 322 to slidably receive tubular member 702. Tubular member 702 would extend through bore 320 and out from plunger 316.

In operation, tubular prosthesis 720 is deployed in vessel V and the distal ends of fastener tubes 706a, b . . . n are positioned slightly beyond the target fixation sites as shown in FIG. 11A. The proximal end of the fastener tubes are held in a fixed position and the proximal end of tubular member 702 retracted as shown in FIG. 11B. As tubular member 702 is retracted, struts 704a, b . . . n are forced radially outward. As struts 704a, b . . . n move radially outward, distal end portions 708a, 708b . . . 708n of fastener tubes 706a, b . . . n, which extend beyond catheter (sheath) 103, move radially outward and close to or contact the inner wall of prosthesis 720. The pusher rods, which are connected to plunger 316 (see FIGS. 4A & B) are then advanced to deploy, for example, fasteners 400a, b . . . n, and fixedly secure prosthesis 720 to vessel V. Struts 704a, b . . . n are sufficiently rigid so as not to flex when the fasteners are deployed. This can be accomplished through material selection or heat treating as would be apparent to one of ordinary skill in the art. For example, the struts can be formed from a different and more rigid material than tube 702 and hingedly coupled to the distal end of tube 702. The struts also can include reinforcement members.

In one variation, prosthesis 200 can replace prosthesis 720, and each fastener tube provided with one or more guide member couplings such as coupling 500a, b . . . n so that the distal ends of the tracking members can be guided to predetermined target sites. Once the distal ends of the fastener tubes are in the desired position, tube 702 can be held stationary relative to the other components so that struts 704a, b . . . n support and stabilize the distal ends of the fastener delivery tubes during fastener deployment.

Referring to FIGS. 12A-B, a method of securing prosthesis 200 to bypass an abdominal aortic aneurysm is shown using fastener delivery apparatus 300. In this example, the prosthesis and apparatus are delivered percutaneously to a femoral artery and advanced to the vicinity of the abdominal aortic aneurysm to be bypassed. It should be understood, however, that this example, but merely is provided for illustrative purposes. Accordingly, other prosthesis configurations can be used to treat different vascular disorders.

The proximal portion of bifurcated stent-graft 200 is positioned below branch vessel BV2 and along the proximal landing between aneurysm A and branch vessel BV2. In this example, vessel V is the aorta and two branch vessels BV1 and BV2, which correspond to the renal arteries, are shown. The prosthesis is deployed and fixedly secured using fastener delivery apparatus 300 as described above (FIG. 12B). After the stent-graft is secured with the fasteners, all catheters are withdrawn. Contralateral leg portion 208, which can include a tubular graft member and annular wire springs or stents 202i-m, is then secured to the graft member short leg portion 206 as is known in the art. The fully deployed stent-graft is shown in FIG. 12B includes ipsilateral leg 204 and contralateral stump 206 to which contralateral leg 208 is coupled using conventional techniques. The combined prosthesis includes stent elements 202a-m.

Any feature described in any one embodiment described herein can be combined with any other feature of any of the other embodiments.

Variations and modifications of the devices and methods disclosed herein will be readily apparent to persons skilled in the art.

What is claimed is:

1. A method of securing a tubular prosthesis to an inner wall of a vessel in a human patient comprising:
endoluminally advancing a tubular prosthesis, having an inner surface, through a vessel in a human body to a target region of the vessel;
endoluminally advancing a plurality of fastener carriers, each carrying at least one fastener, through the vessel to the region; and
deploying the fasteners from the carriers and passing the fasteners from the inner surface of the prosthesis through the prosthesis and the vessel to secure the prosthesis to the vessel.

2. The method of claim 1 wherein the fasteners are simultaneously deployed.

3. The method of claim 1 wherein the vessel is an artery.

4. The method of claim 3 wherein the vessel is the aorta.

5. The method of claim 1 wherein the prosthesis is a tubular graft and the tubular graft surrounds a portion of the fastener carriers.

6. The method of claim 5 including advancing the tubular graft through a catheter and deploying the tubular graft from the catheter.

7. The method of claim 5 including manipulating the fastener carriers to deploy the tubular graft from the catheter.

8. The method of claim 5 including radially expanding said portions of the fastener carriers and the tubular graft.

9. The method of claim 5 including radially expanding said portions of the fastener carriers to radially expand the tubular graft.

10. The method of claim 1 wherein the prosthesis is a bifurcated stent-graft and the fasteners are passed through the vessel in a region between a branch vessel that branches from the vessel and an aneurysm.

11. The method of claim 10 wherein the vessel is the aorta.

12. The method of claim 1 wherein the carriers are tubular members.

13. The method of claim 12 wherein each tubular member is tracked along a flexible guide member disposed inside a catheter.

14. The method of claim 13 wherein each flexible guide member has an end attached to a site on an inner surface of the prosthesis.

15. The method of claim 14 wherein the prosthesis comprises a tubular graft.

16. A prosthesis delivery system comprising:
a tubular prosthesis having an inner wall;
a plurality of guide members extending from said inner wall; and
a plurality of fasteners coupled to one or more of said guide members.

17. The prosthesis delivery system of claim 16 wherein each of said fasteners has a memory shaped loop configuration.

18. The prosthesis delivery system of claim 17 wherein each of said fasteners has a sharp piercing end adapted for piercing through the prosthesis.

19. The prosthesis delivery system of claim 16 further comprising a tubular member slidably coupled to one or more of said guide members, at least one of said fasteners being disposed in said tubular member.

20. The prosthesis delivery system of claim 19 wherein said tubular member has a length of at least about 50 cm.

21. The prosthesis delivery system of claim 19 wherein said fasteners are slidably disposed in said tubular member and further including a pusher member disposed in said tubular member and adapted to push said fasteners out from the tubular member.

22. The prosthesis delivery system of claim 16 comprising a plurality of said tubular members, each being slidably coupled to one of said guide members, each tubular member carrying one of said fasteners.

23. The prosthesis delivery system of claim 22 wherein said tubular members each have a length of at least about 50 cm.

24. The prosthesis delivery system of claim 22 further including a pusher member disposed in each of said tubular members and adapted to push a fastener out from the tube in which it is positioned.

25. The prosthesis delivery system of claim 24 further including a plunger, each of said pusher members being secured to said plunger.

26. The prosthesis delivery system of claim 22 further including an expandable member, said expandable member being surrounded by said tubular members.

27. The prosthesis delivery system of claim 26 wherein said expandable member is an expandable balloon.

28. The prosthesis delivery system of claim 26 further including a tube surrounded by said tubular members, said expandable member comprises a plurality of struts, each being pivotally coupled to said tube and said tubular members.

29. The prosthesis delivery system of claim 28 wherein said struts are rigid.

30. A prosthesis delivery system comprising:
a catheter having a lumen;
a tubular prosthesis having an inner wall surface and being disposed in said catheter lumen;
a plurality of guide members extending from said inner wall surface; and
a plurality of fasteners coupled to one or more of said guide members.

31. The prosthesis delivery system of claim 30 wherein each of said fasteners has a memory shaped loop configuration.

32. The prosthesis delivery system of claim 30 wherein each of said fasteners has a sharp piercing end adapted for piercing through the prosthesis.

33. The prosthesis delivery system of claim 30 further comprising a tubular member slidably coupled to one or more of said guide members, at least one of said fasteners being disposed in said tubular member.

34. Endovascular fastener delivery apparatus comprising:
a catheter having a proximal end and a distal end;
at least one fastener delivery tube disposed in said catheter and having a proximal end portion and a distal end portion;
at least one self-closing fastener disposed in said fastener delivery tube;
and an expander including a radially extendable arm pivotally coupled to said distal end portion of said at least one fastener delivery tube.

35. The apparatus of claim 34 wherein said fastener delivery tube has a length of at least about 50 cm.

36. The apparatus of claim 34 comprising a plurality of said fastener delivery tubes and said expander includes a plurality of said radially expandable arms, each arm being pivotally coupled to one of said fastener delivery tubes along said distal end portion of said tube.

37. The apparatus of claim 36 further including a tubular prosthesis disposed in said catheter in the vicinity of said catheter distal end and distal to said distal end portions of said fastener delivery tubes.

38. The apparatus of claim 36 further including a tubular prosthesis disposed in said catheter in the vicinity of said catheter distal end and surrounding said distal end portions of said fastener delivery tubes.

39. A graft implantation device comprising:
a catheter having a distal end and a proximal end, said distal end including a tubular graft for implantation, a fastener delivery mechanism for delivering at least two fasteners simultaneously and being disposed within said tubular graft,
wherein in a delivery configuration of said catheter, tubular graft, and fastener delivery mechanism have a delivery outside diameter to provide a profile adapted for delivery through the vasculature to a treatment site;
wherein in a pre-deployment configuration of said catheter, tubular graft, and fastener delivery mechanism, said tubular graft is held by said fastener delivery mechanism extended radially to a larger diameter than said delivery outside diameter and against a surrounding tissue wall, where upon actuation said fastener delivery mechanism for delivering at least two fasteners simultaneously delivers said at least two fasteners through said tubular graft and into a surrounding tissue, whereby said at least two fasteners fix said tubular graft to said surrounding tissue.

40. The graft implantation device as in claim 38, further comprising:
at least two flexible guide members attached to at least two distal attachment locations on at least two locations on the inner wall of said tubular graft, the attachment locations being adjacent to at least two fastener delivery sites, such that at least two tubular members of said fastener delivery system which include fasteners to be delivered are configured to include a flexible guide member receiving opening, to cause the ends of said at least two tubular members to be positioned adjacent said at least two distal attachment locations.

41. Graft implantation apparatus comprising:
a catheter having a distal end portion and a proximal end portion, said catheter being sized for delivery through vasculature of a human patient;
a plurality of fasteners;
a plurality of fastener delivery carriers disposed in said catheter, each fastener delivery carrier having a distal end portion, and each fastener delivery carrier carrying at least one of said fasteners;
an expander coupled to said carriers to radially expand said carrier distal end portions; and
a tubular graft surrounding at least a portion of said distal end portion of said delivery carriers and being disposed in said distal end portion of said catheter.

42. The apparatus of claim 40 wherein said expander comprises an expandable balloon and said carrier distal end portions surround said balloon.

43. The apparatus of claim 41 wherein said expander comprises a member surrounded by said carriers, said expander further comprising a plurality of arms, each having a first portion hingedly coupled to one of said carriers and a second portion hingedly coupled to said member such that said arms can move in an outward direction to radially expand said carriers and said tubular graft.
